(19) United States
(12) Patent Application Publication
(10) Pub. No.: US 2010/0217160 A1
(43) Pub. Date: Aug. 26, 2010

(54) Ultrasonic Wave Radiator for Treatment

(76) Inventors: Takayuki Saguchi, Tokyo (JP); Hiroshi Furuhata, Saitama (JP); Toshiaki Abe, Tokyo (JP); Yuichi Murayama, Tokyo (JP); Toshihiro Ishibashi, Kanagawa (JP)

Correspondence Address: BUCHANAN, INGERSOLL & ROONEY PC, POST OFFICE BOX 1404, ALEXANDRIA, VA, 22313-1404, US

(21) Appl. No.: 11/922,090

(22) PCT Filed: May 17, 2006
(86) PCT No.: PCT/JP2006/310286

§ 371 Date: Mar. 28, 2008

(30) Foreign Application Priority Data
Jun. 13, 2005 (JP) 2005-172194

(51) Int. Cl. A61N 7/00

(52) U.S. Cl. 601/2

(57) ABSTRACT

An ultrasonic wave radiator suitable for a cerebral infarction therapy apparatus that is attached to indeterminate curvilinear surface of the scalp of a patient under therapy and dissolves thrombus inside a cerebral blood vessel by outputting ultrasonic vibrations of a plurality of frequencies or an ultrasonic vibration having a wide frequency band. The ultrasonic transducer 20 are arranged on one surface of a flexible sheet 11 in a grid configuration or in other configurations and are bonded thereto and a adhesive layer is provided on the other surface of said sheet 11.

### TECHNICAL FIELD

This invention relates to an ultrasonic wave radiator for treatment, and more specifically, to an ultrasonic wave radiator for treatment that dissolves thrombus by irradiating an ultrasonic wave onto an obstruction part of a blood vessel caused by thrombus, for example, an embolic site by cerebral infarction etc.

### BACKGROUND ART

For medical therapy of cerebral infarction (ischemic stroke), dissolving thrombus that led to cerebral infarction as early in the stage as possible after crisis is considered to be the most effective first selection. It is widely accepted that the sooner the restart of blood flow by dissolving the thrombus, the higher the effect of therapy becomes and the less the subsequent sequelae (dysphasia, paralysis, etc.) becomes.

As thrombolytic agents, urokinase (UK), streptokinase (SK), tissue plasminogen activator (TPA) having high thrombus affinity, etc. are used to dissolve thrombus. It is considered effective to apply such a thrombolytic agent within three hours after the crisis, and results of the therapy to patients show that improvement of symptoms by 30 to 40% has been observed by neurological evaluation at three months after the crisis.

Currently, improvement research of the therapeutic technique by thrombolysis is being carried out principally in two directions below. The first improvement research of the therapeutic technique aims at improvement of a thrombolysis effect in a therapeutic time window that means a stage when a curative effect is expectable, namely, shortening of a thrombolysis time and restoration from penumbra (a state in which cerebral nerve cells are under ischemia). The second improvement research of the therapeutic technique aims at protecting cerebral nerve cells and further extending a time of the therapeutic time.

As a method for enhancing the thrombolysis effect by a thrombolytic agent, for shortening a thrombolysis time, shortening a time from the crisis to recanalization of blood, and for further reducing a dose of the thrombolytic agent from intravenous infusion by drip, there is proposed a method for promoting thrombolysis by irradiating an ultrasonic wave onto the embolic site (a portion in which the thrombus occurred) and utilizing its ultrasonic energy.

As the thrombolysis method using an ultrasonic wave together, the following two methods have been disclosed. That is, U.S. Pat. No. 5,307,816 discloses the catheter ultrasonic irradiation method in which a catheter with an ultrasonic transducer on its point is inserted into blood vessel and an ultrasonic wave is irradiated onto a vicinity of the embolic site or across the embolic site. Moreover, Japanese Laid Open Patent Publication No. 2004-024668 discloses the transcranial ultrasonic irradiation method in which an ultrasonic wave is irradiated toward the embolic site from the surface of the human body.

Here it is known that the ultrasonic probe use in a conventional ultrasonic therapy apparatus for thrombolysis has inconveniences: an ultrasonic irradiation area is narrow; even when the embolic site (portion where thrombus occurred) in the head of a the patient under therapy is found by the ultrasonic apparatus for diagnosis and an ultrasonic irradiation site suitable for thrombolysis is determined, it is difficult to fix the ultrasonic probe toward the irradiation area. Moreover, since the oscillator of the ultrasonic probe is hard, it is difficult to fix the oscillator by tight contact in the ultrasonic irradiation area of the head of the patient under therapy that is an indeterminate curvilinear surface.

It is an object of this invention to provide an ultrasonic wave radiator that solves the above-mentioned problems, that is, having a wide ultrasonic irradiation area, enabling itself to be sufficiently fixed by tight contact to the ultrasonic irradiation area even when it is an indeterminate curvilinear surface, and making it possible to select an ultrasonic transducer being placed at an optimal position according to a site of therapy, and to irradiate an ultrasonic wave of an optimal frequency.

### DISCLOSURE OF THE INVENTION

An ultrasonic wave radiator for treatment according to this invention is an ultrasonic wave radiator for treatment having a structure that one or a plurality of ultrasonic transducers are stuck on one surface of a flexible sheet, and a structure enables them to be brought into close contact with scalp of patient on another surface of said flexible sheet.

The ultrasonic transducers are arranged and stuck on the front surface of the flexible sheet so as to cover a predetermined area in a grid configuration, in a radial configuration, or in other configurations.

Moreover, the ultrasonic transducer can be made up of a piezoelectric ceramic based material. In this case, the ultrasonic transducer is made up of a piezoelectric material of PZT ceramics or other materials. Furthermore, the ultrasonic transducer can also be made up by covering with filler the surrounding of the transducer elements made up of a piezoelectric ceramic based material.

Still moreover, the ultrasonic transducer can be made up of a film of a polymer material having a piezoelectric characteristic. In this case, the ultrasonic transducer can be made up of a film of polyvinylidene fluoride (PVDF).

The ultrasonic wave radiator for treatment is constructed with a plurality of ultrasonic transducers having the same natural frequency. Moreover, the ultrasonic wave radiator for treatment can also be constructed with a plurality of ultrasonic transducers each having a different natural frequency.

Furthermore, when the ultrasonic transducer is made up of a single piezoelectric ceramic based material, flexibility can be given thereto by forming a large number of slits on the surface of the ultrasonic transducer. At this time, the shape of the ultrasonic transducer may be formed such that its thickness is varied continuously.

Still moreover, it is recommendable that regarding the ultrasonic transducer, the whole of the ultrasonic transducer is filled and coated with a filler except for its sticking surface to the flexible sheet.

Even moreover, the ultrasonic wave radiator shall be provided with a cooling device for cooling the ultrasonic transducer. In addition, the ultrasonic wave radiator shall be used only in one time use mode.

### BRIEF DESCRIPTION OF DRAWINGS

FIG. 1 is a diagram illustrating a state in which an ultrasonic wave radiator according to this invention is applied to the head of a patient under therapy A.

FIG. 2 is a perspective view of the ultrasonic wave radiator.

FIGS. 3(a), 3(b) and 3(c) are diagrams illustrating an arrangement state of ultrasonic transducers.

FIG. 4 is a sectional view illustrating a construction that the surrounding of the ultrasonic transducer is filled and coated with a filler.

FIG. 5 is a sectional view illustrating a construction of a single ultrasonic transducer.

FIGS. 6(a), 6(b) and 6(c) are sectional views illustrating a construction of the ultrasonic transducer made up of a film of a polymer material having a piezoelectric characteristic.

FIG. 7 is a diagram illustrating a sectional shape of the ultrasonic wave radiator that is constructed with a plurality of ultrasonic transducers driven at a single frequency.

FIG. 8 is a diagram illustrating a sectional shape of the ultrasonic wave radiator that is constructed with a plurality of ultrasonic transducers driven at a plurality of different frequencies.

FIG. 9 is a sectional view illustrating a construction of the ultrasonic transducer that is formed in a shape such that the thickness of a single ultrasonic transducer is varied continuously.

FIG. 10 is a side view illustrating one example of a cooling device of the ultrasonic wave radiator.

FIG. 11 is a diagram illustrating one example of a use mode of the ultrasonic wave radiator.

FIG. 12 is diagram illustrating wave forms of high frequency currents outputted from high frequency oscillator.

FIG. 13 is a diagram illustrating one example of a state of a continuous sinusoidal wave that was subjected to frequency modulation.

### DETAILED DESCRIPTION OF THE PREFERRED EMBODIMENTS

Hereafter, embodiments of this invention will be described. First, a basic concept of an ultrasonic wave radiator for cerebral infarction therapy will be explained.

### [Basic Concept of Ultrasonic Wave Radiator]

The ultrasonic wave radiator according to this invention is an ultrasonic wave radiator for treatment aiming at dissolving thrombus by emitting an ultrasonic wave toward the thrombus, and is developed by setting it as a goal to become able to be applied to one ultrasonic wave radiator in an inclusive manner with respect to a wide range of a lesion part, from a deep lesion part of the brain to a shallow lesion part, among cerebral blood vessels that are obstructed by thrombus. Since for this purpose it is required for the ultrasonic wave radiator to be applied to a wide area of an indeterminate curvilinear surface of the head of the patient under therapy, being brought into close contact with it, the whole shape shall be formed to be a soft sheet.

Moreover, the ultrasonic wave radiator for treatment that is intended to dissolve thrombus will irradiate an ultrasonic wave transcranially. In doing this, there is a problem that the ultrasonic wave will be attenuated by the cranial bone. The ultrasonic wave has a characteristic that permeability of the cranial bone is improved as its oscillation frequency is reduced. Since the cranial bone is different in thickness and bone mineral density and is not uniform from site to site, it is conceivable that the ultrasonic wave may attenuate depending on its irradiation site and there may be a case where a sufficient irradiation effect cannot be attained. Considering this, if by irradiating an ultrasonic wave of a comparatively low frequency is irradiated to a part of the cranial bone where the thickness of the bone is thick, and an ultrasonic wave of a comparatively high frequency is irradiated to a part of a thin thickness, such as the temporal bone window where the thickness of the bone is thin, it will become possible to solve the problem.

Furthermore, as another problem in irradiating an ultrasonic wave transcranially, there is reflection of the ultrasonic waves inside the cranium. An ultrasonic wave that is irradiated transcranially is reflected on the opposite internal surface of the cranial bone. If phases of the incident wave and of the reflected wave agree with each other, a standing wave occurs and a strong vibration takes place, which may cause impairment to the brain. As a method for circumventing this, by driving the ultrasonic transducer with a drive signal that is a burst wave or a continuous wave of a single frequency subjected to frequency modulation within a time of 1 ms or less, the standing wave can be attenuated or extinguished.

As a method for acquiring the same effect as this, there is a method for irradiating ultrasonic waves of different frequencies simultaneously from one ultrasonic wave radiator.

From these reasons, the ultrasonic wave radiator according to this invention is constructed by mounting a single or a plurality of ultrasonic transducers having different frequency characteristics on a single unit and is configured to be able to avoid a standing wave.

This ultrasonic wave radiator needs for hairs to be shaved as a premise in order to be applied by being brought into close contact with a wide area of the skin of the head (hereinafter referred to as scalp) of the patient under therapy. In this case, since a structure of enabling itself to be brought into contact longitudinally, in order to keep adhesion to the scalp, for example, a layer having adhesion shall be formed on a surface of the ultrasonic wave radiator that contacts with the scalp and the apparatus shall be configured to be adhered directly to the scalp through this layer.

For this reason, the ultrasonic wave radiator shall be disposable, limiting its use as one-time use, from the viewpoint of hygiene standards and stability of the contact surface.

Further, although this ultrasonic wave radiator is connected with an ultrasonic oscillator that is a drive source and an amplifier, it shall establish wire connection through wire for this purpose. The ultrasonic wave radiator is configured to be detachable with the ultrasonic oscillator and the amplifier that are peripheral devices, not constituting an integral construction with the peripheral devices.

In order to prevent heat generation by the use of the ultrasonic wave radiator from affecting the head of the patient under therapy, a cooling device shall be disposed in the circumference of the ultrasonic wave radiator.

Incidentally, as described above, the ultrasonic wave radiator according to this invention is an ultrasonic wave radiator for treatment, not an ultrasonic wave radiator aiming at diagnosis.

### [Constructions of Ultrasonic Wave Radiator and of Ultrasonic Transducer]

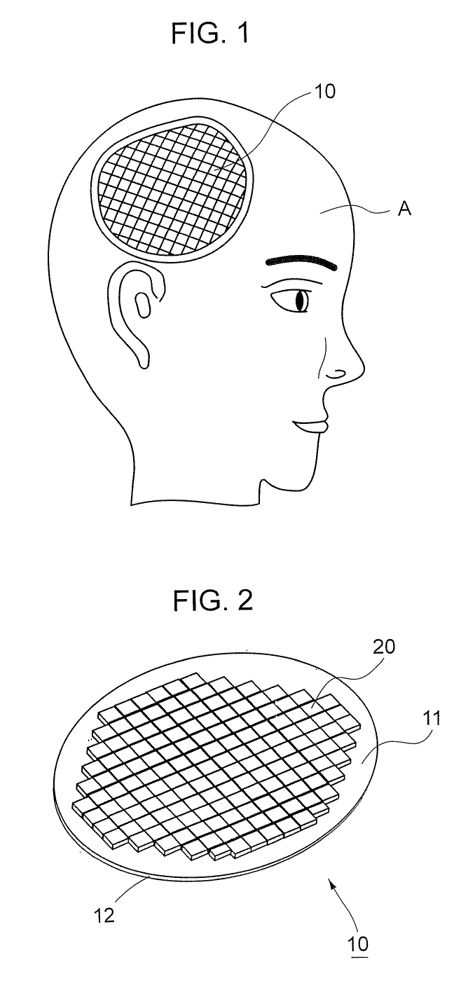

Next, constructions of ultrasonic wave radiator and ultrasonic transducer will be explained. FIG. 1 is a diagram illustrating a state in which the ultrasonic wave radiator according to this invention is applied to the head of the patient under therapy A and FIG. 2 is a perspective view of an ultrasonic wave radiator 10. The ultrasonic wave radiator 10 is constructed with a large number of columnar (meaning that they have a thickness) ultrasonic transducers 20 arranged in a grid configuration and are bonded to one surface of a flexible sheet 11. An adhesive layer 12 is formed on another surface of said flexible sheet which is a contact surface contacting with the scalp of the sheet 11 and is configured to allow itself to be adhered directly to the scalp through the adhesive layer 12. Incidentally, although FIG. 2 shows an example in which the ultrasonic transducers 20 are arranged in a grid configuration, they may be arranged otherwise, i.e., in a radial configuration or other configurations.

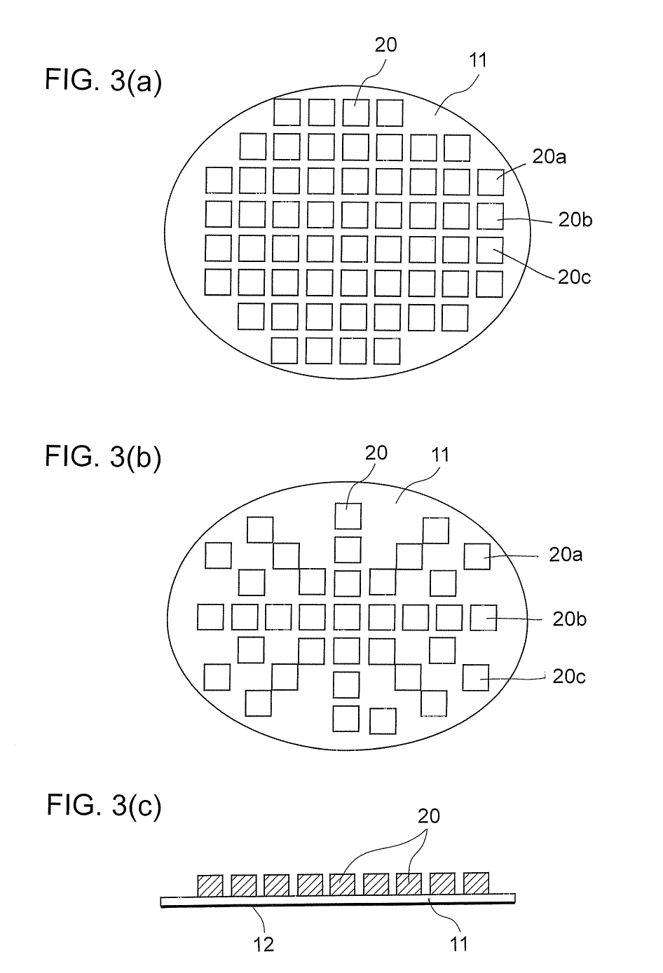

FIGS. 3(a), 3(b), and 3(c) are diagrams illustrating an arrangement state of the ultrasonic transducers 20, wherein FIG. 3(a) shows an example in which a large number of columnar ultrasonic transducers 20 (20a, 20b, - - - ) are arranged in a grid configuration, FIG. 3(b) shows an example in which a large number of columnar ultrasonic transducers 20 (20a, 20b, - - - ) are arranged in a radial configuration, and FIG. 3(c) shows its sectional view. Besides the grid configuration or the radial configuration described above, the ultrasonic transducers 20 may be arranged in other appropriate configuration that is suited to therapy purpose.

By arranging a large number of ultrasonic transducers 20 on the flexible sheet 11, even in the case where the ultrasonic transducer 20 itself is made up of a hard ceramic based material, flexibility can be given to the ultrasonic wave radiator 10.

In addition to this, as a construction of giving flexibility to the ultrasonic wave radiator 10, the ultrasonic transducer 20 that is made up of a complex material is proposed. Here, the complex material designates a construction where the surrounding of the ultrasonic transducer 20 made up of a ceramic based material is filled and coated with a filler, and the like.

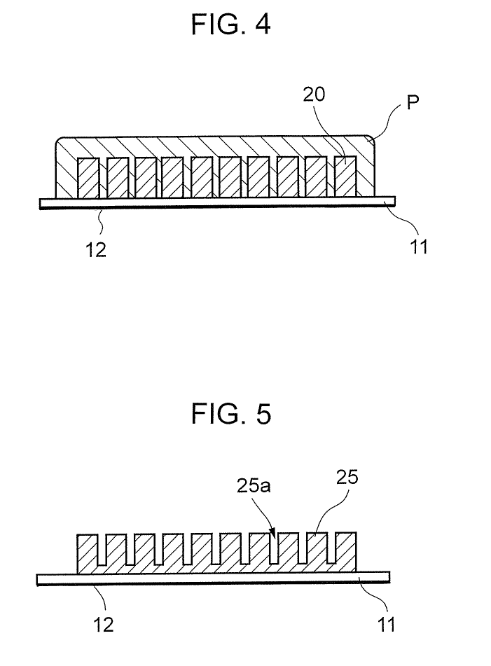

FIG. 4 is a sectional view of what is constructed with a complex material such that the surrounding of a plurality of ultrasonic transducers 20 each made up of a ceramic based material is filled and coated with the filler. The surrounding of the plurality of ultrasonic transducers 20, except for a contact side (adhesion-provided layer 12 side) between the sheet 11 and the scalp, is filled and coated with the filler P that gives bearing properties. According to this construction, the ultrasonic transducers 20 can be protected by the filler P that is filled around the plurality of ultrasonic transducers 20, and the flexibility is not impaired. As the filler P, for example, use of a resin material or gel can be considered. As the resin material, the degree of flexibility given can be adjusted by selecting from among comparatively hard epoxy resins and urethane resins, comparatively soft urethane resins, and gels.

In addition to them, by the ultrasonic transducer 20 being made up of a composite material such that a powder ceramic instead of a hard ceramic based material is mixed into the filler having elasticity, flexibility can be given to the ultrasonic transducer itself.

FIG. 5 is a sectional view illustrating a construction of a single ultrasonic transducer 25, which is constructed by forming a large number of slits 25a on the ultrasonic transducer in a grid configuration or in other configurations and bonding its surface on which these slits 25a are not formed to the sheet 11. According to this construction, flexibility can be given even if the ultrasonic transducer is constructed with a single ultrasonic transducer.

Also in this construction, like the construction described above, the surrounding of the ultrasonic transducer 25 including the slits 25a may be filled with the filler P. According to this construction, it is not only possible to protect the ultrasonic transducer to which flexibility was given by the slits 25a but also flexibility cannot be impaired.

In addition to the above, the ultrasonic transducer 20 can be made up of a film of a polymer material having a piezoelectric characteristic. As a film of a polymer material, polyvinylidene fluoride (PVDF) etc. are conceivable. In the case where the ultrasonic transducer 20 is made up of a film of a polymer material, in order to make it adapted to a comparatively low oscillating frequency, laminating plural sheets of the film can make it adaptable to this.

However, since it is necessary to alter the number of lamination sheets of the film in order to generate a plurality of ultrasonic vibrations each having a different frequency, a plurality of ultrasonic transducers each having a different number of lamination sheets according to an oscillating frequency are made and are bonded to the sheet to construct the ultrasonic transducers. In addition to this, in order to generate an ultrasonic vibration of a different frequency, it is also possible to support it by altering the thickness of the film.

Moreover, in the case of generating the ultrasonic vibration of a single frequency, the following two methods can be adopted: an ultrasonic transducer obtained by laminating a plural sheets of a film of a polymer material just by the number according to an oscillating frequency is bonded to the sheet to make the construction; and a film of a thickness that accords with the oscillating frequency is used to support the requirement. Furthermore, in the case of generating an ultrasonic vibration of a single frequency, the sheet may be omitted and a layer having adhesion may be formed directly on the film of the polymer material that is the lowermost layer.

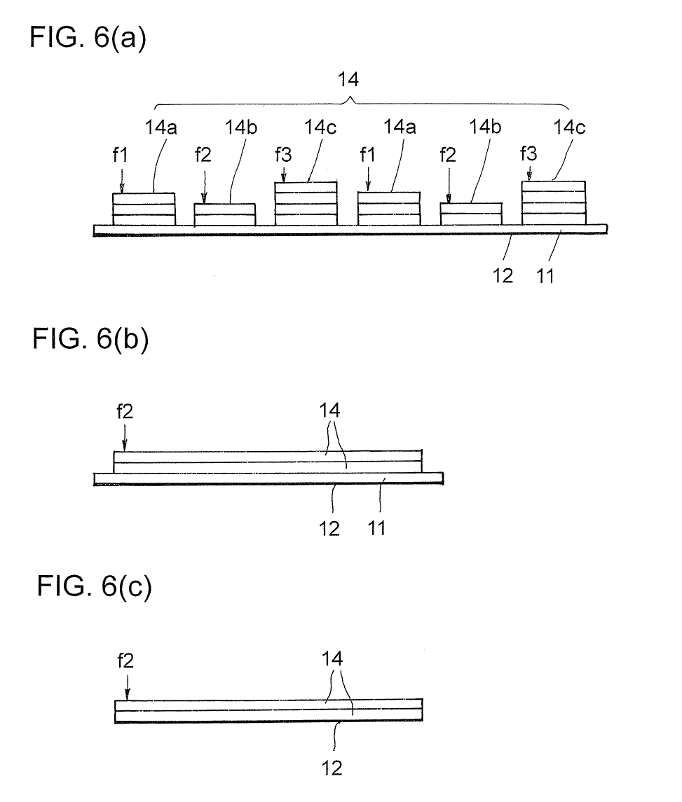

FIGS. 6(a), 6(b), and 6(c) are sectional views illustrating a construction of the ultrasonic transducer made up of a film of a polymer material having a piezoelectric characteristic. FIG. 6(a) is a construction for generating a plurality of ultrasonic vibrations having different frequencies of natural frequencies f1, f2, and f3. This construction is what is constructed by bonding a plurality of ultrasonic transducers 14 each of which differs in the number of lamination sheets, i.e., an ultrasonic transducer 14a of natural frequency f1, an ultrasonic transducer 14b of natural frequency f2, and an ultrasonic transducer 14c of natural frequency f3, to the sheet 11. On the sheet 11, the adhesive layer 12 is provided on the opposite side thereof to the ultrasonic transducer 14.

FIG. 6(b) is a construction for generating an ultrasonic vibration of a single frequency. In this example, the construction is such that the ultrasonic transducer 14 of natural frequency f2 is made and bonded to the sheet 11. On the sheet 11, the adhesive layer 12 is provided on the opposite side thereof to the ultrasonic transducer 14b.

In addition to this, FIG. 6(C) is also a construction for generating an ultrasonic vibration of a single frequency. This example is a construction where the adhesive layer 12 is provided directly on the lowermost layer film of the ultrasonic transducer 14 in which plural sheets are laminated.

Several construction examples of the ultrasonic transducer were explained in the foregoing. In any construction, electrodes shall be formed by means of evaporation of an electrode material and the like on one end face of the ultrasonic transducer and on the other end face opposite to this, and shall be connected to feed terminals.

### [Oscillating Frequency of Ultrasonic Wave Radiator]

Next, oscillating frequencies of the ultrasonic wave radiator will be explained. As described above, the ultrasonic wave radiator 10 is constructed by arranging a plurality of ultrasonic transducers 20 in a grid configuration or in other configurations or is constructed from a single ultrasonic transducer 25 on which the slits are formed. In addition to them, it is made up of a film of a polymer material having a piezoelectric characteristic. An oscillating frequency of the ultrasonic wave radiator is determined by a natural frequency f of the ultrasonic transducer, and the natural frequency f is determined by the thickness of the ultrasonic transducer (in the case where the ultrasonic transducer is columnar, its height does; in the case of a film of a polymer material, the number of lamination sheets of the film and/or the thickness of the film does).

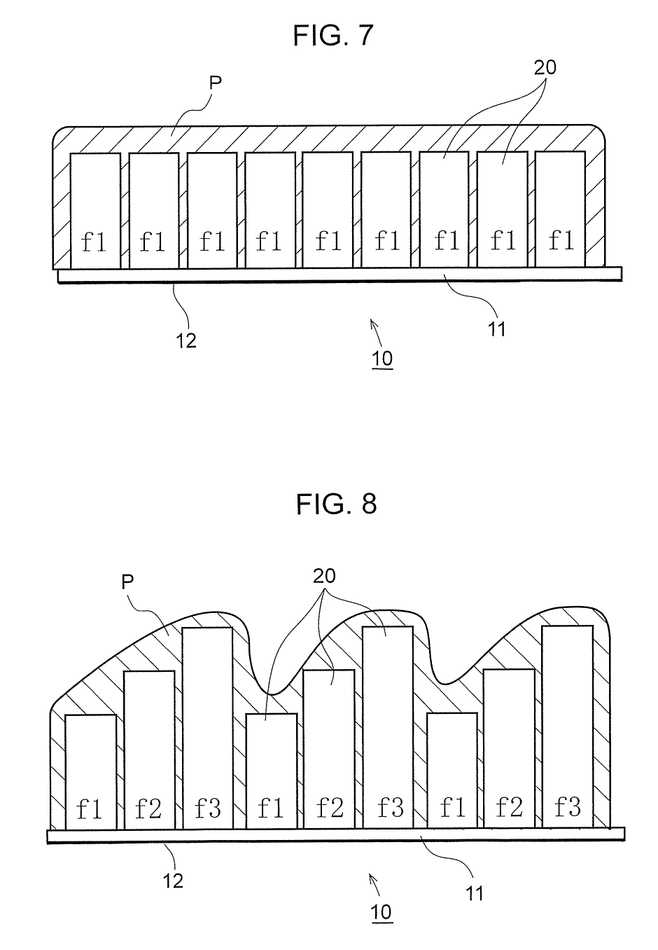

FIG. 7 is a diagram illustrating a sectional shape of the ultrasonic wave radiator 10 that is constructed with a plurality of ultrasonic transducers 20 driven at a single frequency, showing a construction where the ultrasonic transducer 20 of natural frequency f1 is bonded to the sheet 11 and the surrounding of the ultrasonic transducer 20 is filled and coated with the filler P. Since in the ultrasonic wave radiator 10 driven at a single frequency, all the heights of the plurality of ultrasonic transducers 20 become equal, a surface of the ultrasonic wave radiator 10 opposite to the sheet 11 will be substantially planar surface. The adhesive layer 12 is provided on the rear surface of the sheet 11.

FIG. 8 is a diagram illustrating a sectional shape of the ultrasonic wave radiator 10 that is constructed with a plurality of ultrasonic transducers 20 driven at a plurality of different frequencies, showing a construction where the ultrasonic transducers 20 of the natural frequencies f1, f2, and f3 are bonded to the sheet 11, and the surrounding of the ultrasonic transducers 20 are filled and coated with the filler P. The adhesive layer 12 is provided on the rear surface of the sheet 11. Since in the ultrasonic wave radiator 10 driven at a plurality of different frequencies, heights of the plurality of ultrasonic transducers 20 differ, the surface of the ultrasonic wave radiator 10 opposite to the sheet 11 become a plane having unevenness. Incidentally, the size in a height direction is shown, being exaggerated for explanation in FIG. 7 and FIG. 8.

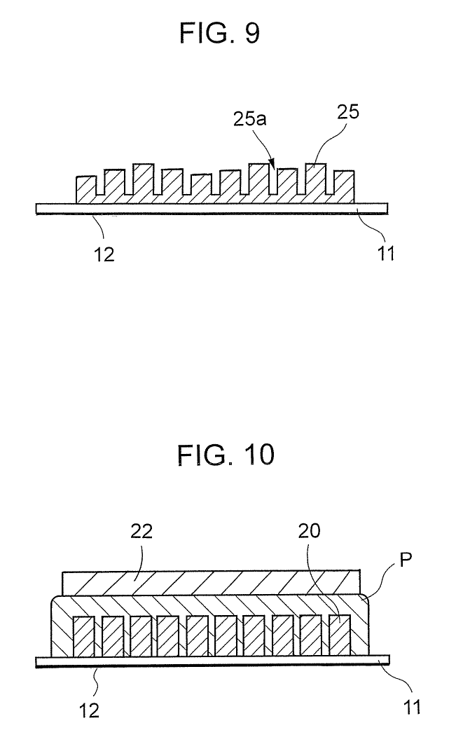

FIG. 9 is a sectional view illustrating the construction of the ultrasonic transducer that is formed in a shape such that the thickness of a single ultrasonic transducer 25 shown in the above-mentioned FIG. 5 is continuously varied. With the configuration shown in FIG. 5, the ultrasonic transducer is driven at a single frequency and only an ultrasonic vibration at a single frequency can be outputted. By adopting the configuration shown in FIG. 9, it is possible to generate ultrasonic vibrations of a plurality of frequencies with a single ultrasonic transducer 25 and to output an ultrasonic vibration having a wide frequency band as a whole.

Regarding the ultrasonic transducer made up of the film of a polymer material having a piezoelectric characteristic, its oscillating frequency was explained previously in the explanation of the construction of the ultrasonic transducer referring to FIG. 6(a) to FIG. 6(c), and so the explanation is omitted here.

A reason of outputting ultrasonic vibration of a plurality of frequencies using the plurality of ultrasonic transducers 20 and a reason of making a single ultrasonic transducer 25 output an ultrasonic vibration having a wide frequency band are: to make it possible to become able to use an ultrasonic vibration of a frequency at which decrement is comparatively small according to a site of irradiation because an irradiated ultrasonic vibration becomes different depending on the thickness of the cranial bone at the site of irradiation that the irradiated ultrasonic vibration encounters, as explained in the fundamental concept of the ultrasonic wave radiator - - - ; and to make a standing wave that arises by reflection against the inner surface of the cranial bone attenuate or extinguish.

### [Constituent Material of Ultrasonic Transducer]

Materials that constitutes the ultrasonic transducer will be explained. The first material is a hard ceramic based material. A material currently used widely is (Pb(Zr, Ti)O₃), called PZT, that is a solid solution of Pb, TiO, and PbZrO₃. Since lower the frequency of the ultrasonic transducer, thicker the thickness thereof becomes, when the ultrasonic transducer is driven at a low frequency, if it is constructed with a hard ceramic based material, it becomes disadvantageous in terms of flexibility. In this invention, as described above, the construction becomes compatible with the flexibility by arranging a large number of ultrasonic transducers in a grid configuration or in other configurations or, in the case of a single ultrasonic transducer, by providing a large number of slits thereon.

The second material is a composite raw material such that a plurality of PZT elements are covered with the filler having elasticity, for example, a resin material. This material can be used to construct the ultrasonic transducer in which coverage of the filler having elasticity can give flexibility to the ultrasonic transducer itself.

The third material is a film of a polymer material having a piezoelectric characteristic, and includes, for example, polyvinylidene fluoride (PVDF). In order to adapt it to an oscillating frequency, the ultrasonic transducer is constructed by laminating plural sheets of PVDF films. Since the raw material is a film, it excels in flexibility.

### [Cooling of Ultrasonic Transducer]

The ultrasonic transducer generates heat by being supplied a high frequency current. Moreover, the cranial bone of the patient under therapy A to which an ultrasonic wave was irradiated generates heat by absorption of the ultrasonic vibration. Since there is a possibility that the heat generation of such ultrasonic transducers and the heat generation of the cranial bone exert a detrimental effect to the brain tissue, it is necessary to cool them down. To do this, the cooling device is provided in the ultrasonic wave radiator. For its site, it is considered as one example that it is disposed between the ultrasonic transducer and the scalp of the patient under therapy A.

There are a plurality of measures as the cooling device. FIG. 10 is a side view illustrating a first example of the cooling device of the ultrasonic wave radiator, in which a support member 22 for supporting the ultrasonic transducer is disposed on an end face opposite to an ultrasonic irradiation face of the ultrasonic transducer and the support member 22 itself is given a structure having a radiation effect. As structures having radiation effects, there are an air cooling structure, a water cooling structure, a structure with an internal endothermic material, arranging a Peltier element disposed in the support member 22, etc.

Alternatively, as other means of the cooling device of the ultrasonic wave radiator, attaching a cooling jacket for cooling by supplying cooling air or cooling water to the ultrasonic wave radiator can also attain cooling. Alternatively, the cooling jacket made up of a tough synthetic resin film etc. filled with a cooling gel may be used. That is, it is cooled to a predetermined low temperature in advance, and at the time of ultrasonic irradiation therapy is disposed between the ultrasonic wave radiator and the skin of the head of the patient under therapy.

### [Use Mode of Ultrasonic Wave Radiator]

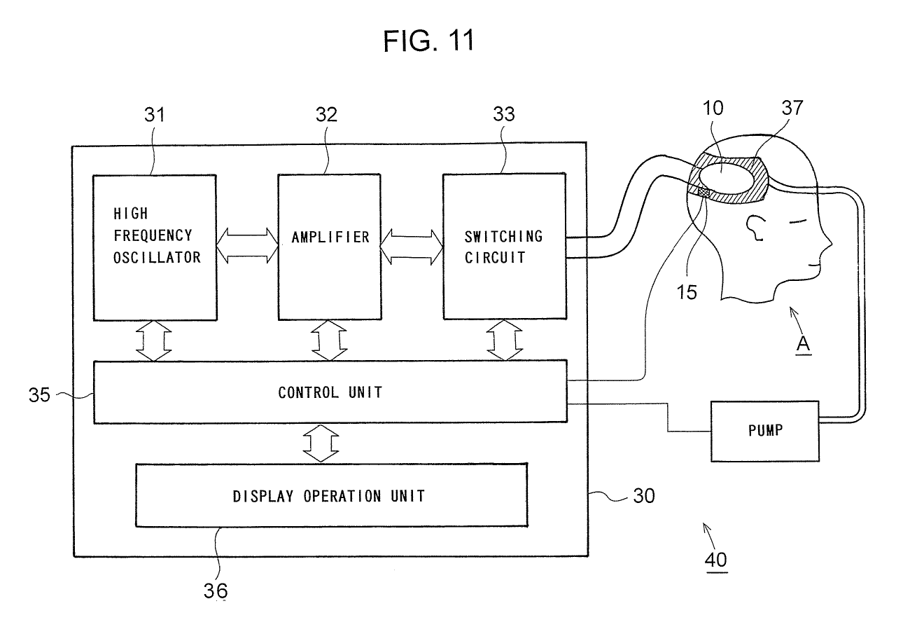

A use mode of the ultrasonic wave radiator according to this invention will be explained briefly. FIG. 11 is a diagram illustrating one example of the use mode of the ultrasonic wave radiator. The ultrasonic wave radiator 10 is stuck on the scalp near the site of therapy of the patient under therapy A that was detected by an ultrasonic diagnostic apparatus prepared in advance separately (not illustrated), and is connected to a control device 30 of an ultrasonic therapy apparatus 40 that the ultrasonic wave radiator 10 according to this invention can use. Moreover, a cooling device 37 (hear, the cooling jacket for circulating cooling water) and a temperature sensor 15 are provided as an adjunct to the ultrasonic wave radiator 10. Incidentally, since the ultrasonic therapy apparatus 40 and the control device 30 are not subjects of this invention, their detailed explanations are omitted.

The control device 30 includes a high frequency oscillator 31 for outputting a high frequency current for driving the ultrasonic transducer 20, an amplifier 32, a switching circuit 33 for selecting a specific ultrasonic transducer to be excited (for example, 20a, 20b, 20c, . . . in FIG. 3) from among the plurality of ultrasonic transducers 20, a control unit 35 for controlling a drive frequency, intensity, a drive time, etc. of the ultrasonic transducer 20, and an operation panel 36, and controls an operation of the ultrasonic therapy apparatus 40.

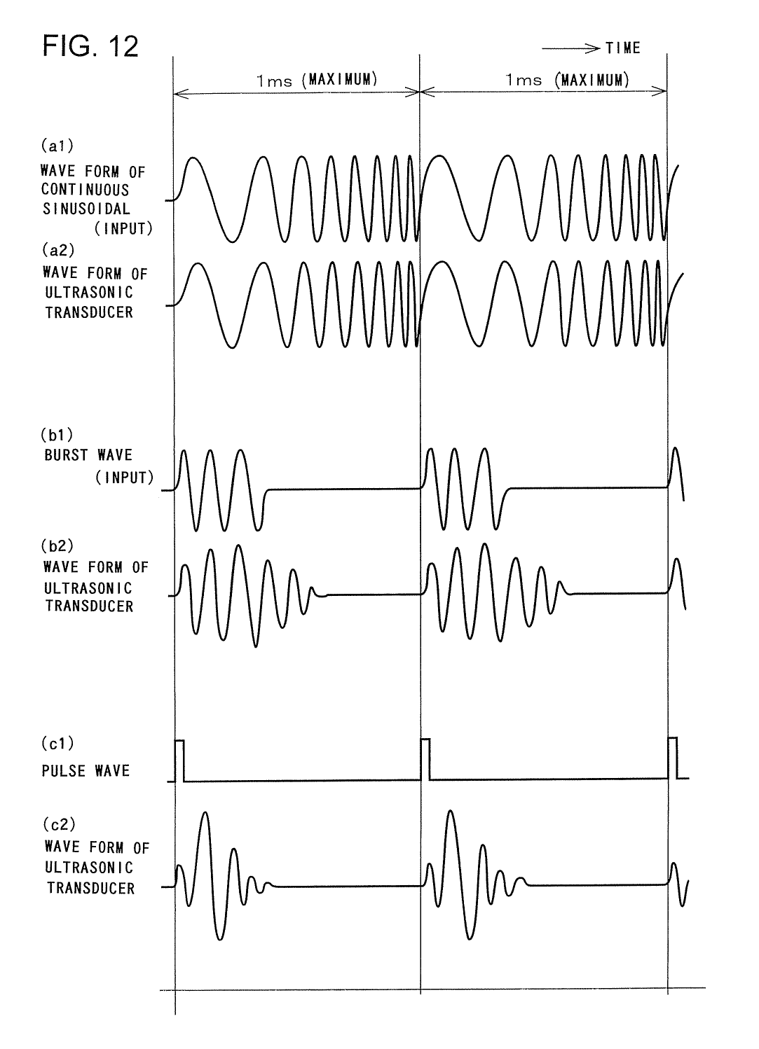

Wave forms of high frequency currents outputted from the high frequency oscillator 31 will be explained. FIG. 12 is diagrams illustrating wave forms of high frequency currents. A continuous sinusoidal wave shown in FIG. 12(a1), a burst wave shown in FIG. 12(b1), a sinusoidal wave intermitting for a predetermined time repeatedly), and a pulse wave shown in FIG. 12(c1) are used.

In the case of the continuous sinusoidal wave, as shown in FIG. 12(a1), frequency modulation is performed in such a way that its frequency is varied periodically. This is because when an ultrasonic wave is irradiated from the outside of the cranial bone at the same frequency continuously, an ultrasonic beam irradiated into the cranial bone from one side of the outside of the cranial bone reflects on an internal surface of the other side of the cranial bone, and the irradiation beam and the reflected beam interfere to form a standing wave inside the cranium, which may cause a local increase of acoustic pressure leading to breeding and impair nerve cells. In the case of the continuous sinusoidal wave, performing frequency modulation can avoid the formation of a standing wave by interference between the irradiation beam and the reflected beam.

Although for the continuous sinusoidal wave, a suitable frequency deviation width is determined without limiting its fundamental frequency, a frequency modulation speed shall be a speed of 1 Hz/ms, namely 1 kHz/S or more. This speed is determined from a critical time in which a standing wave does not occur inside the cranium by ultrasonic irradiation, i.e., a critical time in which cavitation does not arise.

When the ultrasonic transducer is driven by a continuous sinusoidal wave that was subjected to frequency modulation shown in FIG. 12(a1), an ultrasonic vibration of the wave form as shown in FIG. 12(a2) will occur, causing irradiation of an ultrasonic wave.

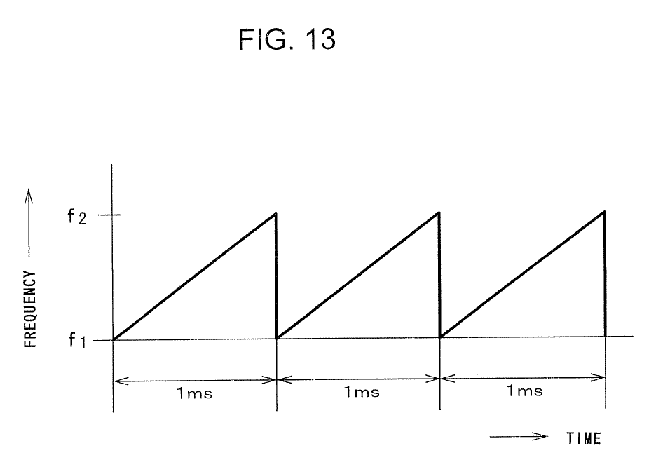

FIG. 13 is a diagram illustrating one example of a state of the continuous sinusoidal wave that was subjected to frequency modulation in which a unit time is set to 1 ms, namely, a repetition cycle is set to 1 ms or less. During this unit time, the frequency varies from f1 to f2, and the frequency returns again to 11; in the next unit time, the frequency varies from f1 to f2.

In the case of a burst wave, the formation of a standing wave inside the cranium can be avoided by setting a duration to 1 millisecond (1 ms) or less, as shown in FIG. 12(b1). When the ultrasonic transducer is driven by a burst wave shown in FIG. 12(b1), an ultrasonic vibration of a wave form as shown in FIG. 12(b2) will occur, and will irradiate an ultrasonic wave.

In the case of a pulse wave, the formation of a standing wave inside the cranium can be avoided by setting the duration to 1 millisecond (1 ms) or less, as shown in FIG. 12(c1). When the ultrasonic transducer is driven by a pulse wave shown in FIG. 12(c1), an ultrasonic vibration of a wave form as shown in FIG. 12(c2) will occur, and will irradiate an ultrasonic wave.

Note that, the average output intensity of a high-frequency signal outputted from the high-frequency oscillator 31 shall be set to 1 W/cm2 in average acoustic intensity in any case of a continuous sinusoidal wave, a burst wave, or a pulse wave.

The ultrasonic wave radiator according to this invention explained in the foregoing is an ultrasonic wave radiator used for an ultrasonic therapy machine aiming at dissolution of embolic site caused by the thrombus that became a cause of cerebral infarction. This ultrasonic wave radiator can be used also for various kinds of therapy purposes such that ultrasonic irradiation may attain a therapy effect except for such therapy of cerebral infarction.

Since the ultrasonic wave radiator of this invention has a structure that enables one or a plurality of ultrasonic transducers to be stuck on the surface of a flexible sheet, and enables the rear surface of the sheet to be brought into close contact with a contact surface of the human body longitudinally, when the ultrasonic apparatus for diagnosis found the embolic site (a part where thrombus occurred) of the head of the patient under therapy, it is possible to fix the ultrasonic wave radiator in a wide area, including the embolic site, of the head of the patient under therapy A and to drive the ultrasonic transducer by selecting it suitable for irradiating an ultrasonic wave to the embolic site.

Then, the ultrasonic transducer can be constructed with the followings: a piezoelectric ceramic based material, for example, a piezoelectric material of PZT ceramics; a piezoelectric material made up of a vibration element of a piezoelectric ceramic based material that is mixed into the filler, for example, a resin material; a film of a polymer material having a piezoelectric characteristic, for example, polyvinylidene fluoride (PVDF); and other films. Then, in either case, in the ultrasonic transducer, the transducer is made up of small elements, or the slits are formed on a large element, or the like, so that these elements are arranged in a grid configuration, a radial configuration, or other configurations so as to cover a predetermined area and are stuck on a sheet, and accordingly the transducer is constructed to have flexibility, whereby the ultrasonic transducer can be brought into close contact with an indeterminate curvilinear surface, such as the head of the patient under therapy A and can be attached on the human body surface stably.

Further, when the ultrasonic transducer is constructed with a plurality of ultrasonic transducers whose natural frequencies are different, an optimal ultrasonic transducer is selected according to a site of therapy, and an ultrasonic wave of an optimal frequency is irradiated, whereby a therapeutic effect can be enhanced.

### INDUSTRIAL APPLICABILITY

This invention is an ultrasonic wave radiator used for an ultrasonic therapy apparatus aiming at dissolution of the embolic site caused by thrombus that became a cause of cerebral infarction of the patient under therapy.

1. An ultrasonic wave radiator for treatment having a structure that one or plurality of ultrasonic transducers are stuck on one surface of a flexible sheet and a structure enables the ultrasonic transducers to be brought into close contact with a scalp of patient on another surface of said flexible sheet.

2. The ultrasonic wave radiator for treatment according to claim 1, wherein said ultrasonic transducers are stuck on the front surface of the flexible sheet in a grid configuration, a radial configuration, or other configurations.

3. The ultrasonic wave radiator for treatment according to claim 1, wherein said ultrasonic transducers are made up of a piezoelectric ceramic based material.

4. The ultrasonic wave radiator for treatment according to claim 3, wherein said ultrasonic transducers are made up of a piezoelectric material of PZT ceramics.

5. The ultrasonic wave radiator for treatment according to claim 1, wherein said ultrasonic transducers are constructed by covering with filler the surrounding of said transducer element made up of a piezoelectric ceramic based material.

6. The ultrasonic wave radiator for treatment according to claim 1, wherein said ultrasonic transducers are made up of a film of a polymer material.

7. The ultrasonic wave radiator for treatment according to claim 6, wherein said ultrasonic transducers are made up of a film of polyvinylidene fluoride (PVDF).

8. The ultrasonic wave radiator for treatment according to claim 1, wherein said ultrasonic transducers are constructed with a plurality of ultrasonic transducers having the same natural frequency.

9. The ultrasonic wave radiator for treatment according to claim 1, wherein said ultrasonic transducers are constructed with a plurality of ultrasonic transducers having different natural frequencies.

10. The ultrasonic wave radiator for treatment according to claim 3, wherein said ultrasonic transducer is made up of a single piezoelectric ceramic based material, a large number of slits are formed on the surface of said ultrasonic transducer, giving it flexibility.

11. The ultrasonic wave radiator for treatment according to claim 1, wherein said ultrasonic transducer is made up of a single piezoelectric ceramic based material, the ultrasonic transducer is formed in a shape whose thickness varies continuously.

12. The ultrasonic wave radiator for treatment according to claim 1, wherein said ultrasonic transducer is filled and coated with the filler except for sticking surfaces thereof to the flexible sheet.

13. The ultrasonic wave radiator for treatment according to claim 1, wherein a cooling device for cooling said ultrasonic transducer is provided as an adjunct thereof.

14. The ultrasonic wave radiator for treatment according to claim 1, wherein it is used only in one time use mode.
